(19) United States
(12) Patent Application Publication
(10) Pub. No.: US 2021/0115832 A1
(43) Pub. Date: Apr. 22, 2021

(54) System And Method For Monitoring Exhaust Gas

(71) Applicant: Johnson Matthey Catalysts (Germany) GmbH, Redwitz an der Rodach (DE)

(72) Inventor: Dirk Reichert, Redwitz an der Rodach (DE)

(21) Appl. No.: 17/074,804

(22) Filed: Oct. 20, 2020

(30) Foreign Application Priority Data
Oct. 22, 2019 (GB) 1915285.9

(51) Int. Cl. F01N 11/00

(52) U.S. Cl. CPC F01N 11/002; F01N 2560/08; F01N 2560/026; F01N 2560/021

(57) ABSTRACT

The present invention relates to a system and method for monitoring the concentrations of various species in exhaust gas expelled from a stationary source. The system comprises an exhaust passage and monitoring apparatus for monitoring exhaust gas, the monitoring apparatus comprising: a sampling tube extending into the exhaust passage for collecting a sample of exhaust gas; a sampling cavity in communication with the sampling tube; and one or more sensors for measuring the concentration of one or more airborne species within the sampling cavity, wherein the monitoring apparatus comprises suction generating apparatus in communication with the sampling cavity for drawing exhaust gas along the sampling tube into the sampling cavity. The invention further relates to a monitoring apparatus for attachment to an exhaust passage of a stationary source and a method of fitting the apparatus to an exhaust passage.

The present invention relates to a system and a method for monitoring the concentrations of various species in exhaust gas expelled from a stationary source such as an exhaust gas expelled from a power plant, gas turbine, or other stationary engine used for generating electricity, or exhaust gas expelled from an industrial process, for example, exhaust expelled from an industrial petrochemical process or the like. In particular, the disclosed embodiments can enable the measurement of the concentration of at least NOx, CO, NH₃, and/or unburnt fuel components such as hydrocarbons (HC) in exhaust gas.

Power plants often utilize fossil fuels as the energy source, such as coal, oil or natural gas, and combustion of these fuels generates exhaust gas that must be treated to remove nitrogen oxides (NOx), including NO (nitric oxide), NO₂ (nitrogen dioxide), and N₂O (nitrous oxide) as well as, carbon monoxide (CO), unburned hydrocarbons (HC) and particulate matter, such as soot. The exhaust generated in power plants is generally oxidative, and the NOx needs to be reduced selectively with a catalyst and a reductant, which is typically ammonia. The process, known as selective catalytic reduction (SCR), was extensively investigated in the 1970s for removing NOx from power plant exhaust gas and the like.

Coal and oil contain various amounts of sulfur. Treatment of exhaust from these plants using SCR demands maintenance of a relatively high NOx reduction efficiency while minimizing SO₂ oxidation. Many SCR catalysts are effective in converting NOx to nitrogen and water in the presence of ammonia. However, an undesirable side reaction, the oxidation of SO₂ to SO₃, commonly occurs along with NOx reduction. The formation of sulfur trioxide (SO₃), a component of acid rain, needs to be controlled.

It is beneficial to be able to monitor the amounts of these species present in exhaust gases. In particular, an assessment of the levels of at least one of NOₓ, CO, NH₃, O₂, SOₓ and HC are important. This can assist in the control of an exhaust gas treatment process or even, in the case of a stationary engine, the combustion process itself.

U.S. Pat. No. 5,703,299 describes an exhaust stack stream sensor having a main hollow pipe with a plurality of port holes therethrough and spaced apart therealong, the main hollow pipe having two spaced apart closed off ends, the main hollow pipe positionable across the interior of an exhaust stack from which flows an exhaust stream, and a sample collecting tube having a first end in fluid communication with an interior of the main hollow pipe and a second end in fluid communication with vacuum apparatus for drawing a portion of the exhaust stream through each port hole, into the main hollow pipe, and through the sample collecting tube for transmission therefrom of a composite sample to additional apparatus.

KR 20 2014 0004209 describes an exhaust gas measuring device for measuring the nitrogen oxide concentration of an exhaust gas passing through an exhaust duct comprising: a probe which is installed in the exhaust duct and has a plurality of suction holes formed radially; a diverting pipe which is connected to the probe and diverts the exhaust gas sucked in through the suction holes to the exhaust duct; a measurement chamber which is formed in the diverting pipe; a concentration sensor which is inside the measurement chamber and measures the nitrogen oxide concentration of the exhaust gas; and a vacuum generator which provides suction power to the suction holes of the probe using the vacuum formed by the introduction of compressed air. The vacuum generator includes a cooling device for forming the vacuum with cooled compressed air.

For many exhaust gas streams emitted from stationary sources, it is necessary to measure, both accurately and quickly, the concentrations of various airborne species in the exhaust gases emitted therefrom. By “airborne” it is meant any species conveyed by or within the exhaust gases. That is, it includes particulate matter or liquid droplets transported aloft within the exhaust gas, as well as species such as NOₓ which form part of the exhaust gas mixture per se. It is of course essential that the species to be measured can be conveyed to a suitable sensor for measurement and thus are part of or carried by the exhaust gas. In most of the embodiments described herein the airborne species are constituent gases within the exhaust gas.

Conventional exhaust gas monitoring devices for stationary applications provide measurements that vary slowly and so the signals they produce significantly lag behind the quantity being measured. In an automotive context, faster sensors are available, but these are suitable for measuring only small flows of exhaust gas and do not produce signals that achieve sufficient accuracy.

For example, downstream of an SCR catalyst, the measured species may include NOx and NH₃. The measurement of the concentrations of these species enables the calculation of an appropriate amount of NH₃ to be introduced into the exhaust gas upstream of the SCR catalyst in order to effectively reduce the concentration of NOx.

The exhaust gas to be monitored may be generated from stationary sources such as thermal power plants, gas turbines, coal-fired power and cogeneration plants, plant and refinery heaters and boilers used in the chemical and petrochemical industries, furnaces, coke ovens, coffee roasting plants, municipal waste plants, and incinerators. In particular, the exhaust gas may be generated by a power plant, a gas turbine, or other form of combustion engine used for generating electricity or power. That is, preferably the stationary source is a combustion source.

Any such stationary source will have an exhaust system for expelling, and optionally treating, exhaust gas. The exhaust system may comprise one or more catalysts for facilitating the conversion of undesirable species present in the exhaust gas before its emission to the atmosphere. For example, the exhaust may contain combustion products, along with unburnt fuel components and/or particulate matter. These airborne species should be monitored in a way that can assist with the control of the process from which the exhaust gas derives or the exhaust treatment process. In exhaust systems which employ an SCR catalyst, for example, there is a need for a fast responding, accurate measure of airborne species so that the appropriate amount of reductant to be introduced into the exhaust gas stream can be calculated.

According to the present invention there is provided a system and a method as defined by the claims.

A first aspect of the invention provides an exhaust system for a stationary source, the system comprising an exhaust passage and monitoring apparatus for monitoring exhaust gas, the monitoring apparatus comprising: a sampling tube extending into the exhaust passage for collecting a sample of exhaust gas; a sampling cavity in communication with the sampling tube; and one or more sensors for measuring the concentration of one or more airborne species within the sampling cavity, wherein the monitoring apparatus comprises suction generating apparatus in communication with the sampling cavity for drawing exhaust gas along the sampling tube into the sampling cavity.

A second aspect of the invention provides a method of monitoring exhaust gas from a stationary source, comprising: collecting a sample of exhaust gas from an exhaust passage of the stationary source; delivering the sample of exhaust gas to a sampling cavity; and measuring the concentration of one or more airborne species within the sampling cavity using one or more sensors, wherein the method is characterised by drawing exhaust gas along the sampling tube into the sampling cavity.

A third aspect of the invention provides a monitoring apparatus for attachment to an exhaust passage of a stationary source, comprising: a body having a first side and a second side, the body arranged to be located over an aperture with the first side facing the aperture and the second side facing away from the aperture; a sampling tube for collecting a sample of exhaust gas, extending from the first side of the body; a sampling cavity on the second side of the body, the sampling cavity in communication with the sampling tube; one or more sensors for measuring the concentration of one or more airborne species within the sampling cavity; and suction generating apparatus on the second side of the body and arranged to draw exhaust gas along the sampling tube into the sampling cavity.

The system and method are suitable for use with stationary sources of exhaust gas. For example, the system and method may be used with a stationary source for generating electricity, including gas turbines and conventional power generation plants. Such a stationary source will typically have an exhaust passage, such as a pipe or stack, having a cross-sectional width or dimension diameter exceeding 25 cm, larger sources even exceeding 1 meter.

It should be noted that exhaust gases are typically at an elevated temperature, especially where they are obtained from a combustion source. However, in other embodiments, such as assessing the species in heating ventilation AC systems (HVAC), the gas may not be at an elevated temperature. In preferred embodiments, the entrainment gas is heated (preferably, up to the temperature of the exhaust gas). This would seem counter-intuitive, because the entrainment effect of such a flow of gas would be weaker than if it were at a lower temperature. For example, a cooled stream of entrainment gas would provide a greater pressure drop. However, it has been found that a heated flow of gas can avoid condensation in the entrainment passage. For example, when a venturi tube is used to entrain sampled exhaust gas, condensation in the venturi tube can be lessened or avoided by the use of heated entrainment gas.

A span gas is a calibration gas or gas mixture used as comparative standard in the calibration of analytical instruments, like gas analysers or gas detectors. Therefore, a calibration gas has to be of a precisely defined nature or composition, for example 500 ppm carbon monoxide in nitrogen. The specific nature of the span gas used will depend on the requirements of the sensor being calibrated and the sensitivity of the emissions being measured.

Since sensors may have an offset or drift in their concentration reading over time, a span gas can be used calibrate the read-out concentration signal. The span gas has a known concentration of the species to be calibrated, typically in the relevant concentration range that this species is anticipated to be in the exhaust gas. For example, in one embodiment for application of a stationary diesel engine, one span gas may have zero another may have 1500 parts per million of NOx. For a gas engine the span gas concentration may be small fraction of aforementioned concentration.

The use of span gas to calibrate the sensors of the monitoring apparatus during use is unusual. However, it enables the use of different types of sensor, such as sensors normally used in automotive exhaust monitoring systems. Such automotive sensors are normally only calibrated during manufacture, but have a factory offset and concentration readings that drift over time. This is not a problem in an automotive context. However, in the context of stationary sources for electrical power generation, emission requirements should be controlled more accurately. It is therefore beneficial to calibrate the sensors more frequently. By calibrating the sensors intermittently during use of the exhaust system, it is possible to use sensors that produce readings that would otherwise drift unacceptably during use.

The terms unburned fuel and unburned hydrocarbons (HC) are used synonymously herein and include hydrocarbons, including CH₄, soot and its precursors.

For a better understanding of the invention and to show how the same may be put into effect, reference will now be made, by way of example only, to the accompanying drawings in which:

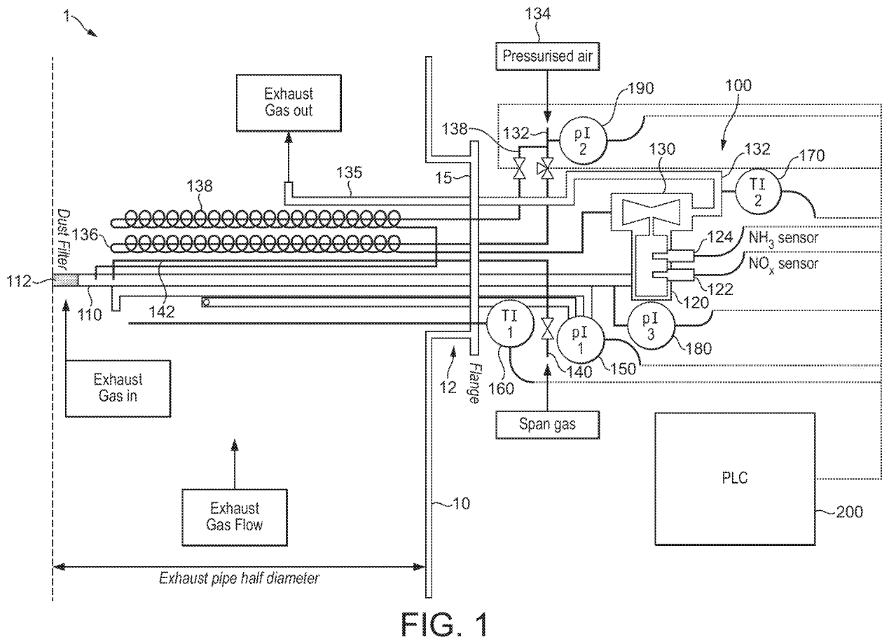

FIG. 1 shows a schematic representation of an embodiment of an exhaust system for a stationary source; and

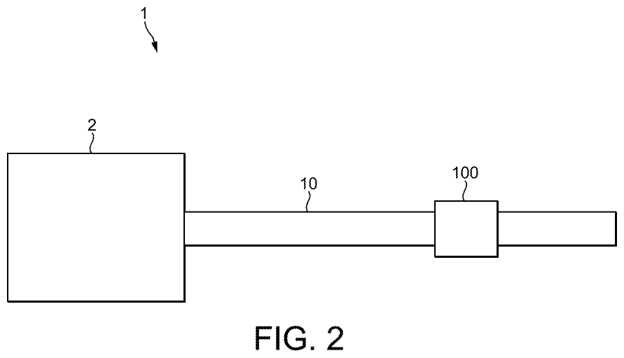

FIG. 2 shows a schematic representation of a stationary source comprising the exhaust system of FIG. 1.

As can be seen from FIG. 1, a preferred embodiment of an exhaust system 1 for a stationary source 2 (shown in FIG. 2) in accordance with the invention comprises an exhaust passage 10 and a monitoring apparatus 100 for monitoring exhaust gas.

The monitoring apparatus 100 comprises: a sampling tube 110; a sampling cavity 120; one or more sensors 122, 124 (for example, chemical sensors); a source of entrainment gas 134; an entrainment passage 132; and, optionally, a source of span gas 140.

Although an entrainment system is shown in this embodiment, any apparatus for generating suction, such as a pump, may be provided to draw exhaust gas along the sampling tube 110.

The sampling tube 110 extends into the exhaust passage 10. In this way, the sampling tube 110 is configured to collect a sample of exhaust gas from the exhaust passage 10. For example, the sampling tube 110 may have one or more holes 112, such as a hole at its distal end, and/or a plurality of holes spaced along its length. Optionally, a dust filter for all or each hole is arranged for preventing dust in the exhaust gas from entering the sampling tube 110.

The sampling tube 110 is particularly beneficial for larger stationary sources. The sampling tube 110 may be for example at least 200 mm in length. Such a tube is particularly useful for sampling from the middle of an exhaust passage 10 of 400 mm. More preferably, the sampling tube 110 is at least 400 mm in length.

The sampling cavity 120 is in communication with the sampling tube 110.

The one or more sensors 122, 124 are configured to measure the concentration of one or more airborne species within the sampling cavity 120. Preferably, these are calibrated to measure at the temperature of the exhaust gas. For example, one sensor 122 may be provided to sense one or more airborne species, a plurality of sensors may be provided to sense respective airborne species.

The sensors may be arranged to sense one or more of: NOx; CO, unburned hydrocarbons (HC) (including CH₄, soot and its precursors), SOₓ and NH₃. For example, there may be an NOx sensor 122 (which may measure both O₂ and NOx), and an NH₃ sensor 124. There may be one or more sensors for detecting unburnt species resulting from incomplete combustion and/or slip fuel (for example, solid state sensors are available for this purpose). There may be a sensor for acidic species, such as SOₓ. Accurate measurement of one or more of these species may assist with emission limit compliance. Accurate measurement of the NOx level permits accurate dosing of NH₃ into the exhaust gas to facilitate NOx treatment on a downstream SCR catalyst. Accurate measurement of the NH₃ level downstream of an SCR catalyst can be used as feedback to prevent over-dosing of the NH₃. The SCR catalyst and means for dosing NH₃ into the exhaust gas are well known in the art and are not shown. The measurement of unburnt species can indicate insufficiencies in combustion and, if installed downstream of an oxidation catalyst, indicate aging of the oxidation catalyst.

In addition to the sensors 122, 124, it is preferable that the monitoring apparatus 100 comprises an exhaust gas temperature sensor 160. This can be used for referencing emission values to the relevant emission reporting reference state. The exhaust gas temperature sensor 160 preferably includes a probe extending into the exhaust passage 10.

Optionally, the monitoring apparatus 100 also comprises an exhaust gas pressure sensor 150. The exhaust gas pressure sensor 150 preferably includes a pitot tube for measuring the static and dynamic pressure in the exhaust passage 10 proximate to the tip of the sampling tube 110. This can be used to calculate volumetric flow of the exhaust gas for accurate calculation of the ammonia dosing amount for the denitrification reaction.

Optionally, the monitoring apparatus 100 also comprises a sample gas pressure sensor 180. The sample gas pressure sensor 180 preferably measures the pressure of the sampled exhaust gas in the sampling tube 110. For example, the sample gas pressure sensor 180 preferably measures the pressure of the sampled exhaust gas immediately upstream of the sample cavity 120. This can be used to ensure the volumetric flow over the sensors is within the sensor-specific limits for accurate concentration measurement.

Optionally, the monitoring apparatus 100 also comprises an entrainment gas pressure sensor 190. The entrainment gas pressure sensor 190 measures the pressure of the source of entrainment gas 134. This can be used to control the suction pressure of the venturi nozzle.

Optionally, the monitoring apparatus 100 also comprises an entrained gas temperature sensor 170. The entrained gas temperature sensor 170 preferably measures the temperature pressure of the combined flow of sampled exhaust gas entrained into the flow of entrainment gas. For example, the entrained gas temperature sensor 170 preferably measures the temperature of the combined flow of sampled exhaust gas and entrainment gas immediately downstream of the sample cavity 120. The entrained gas temperature 170 can provide a measurement of the temperature of the combined flow before reintroduction into the exhaust passage 10. This can be used to keep the volume flow in the sampling cavity 120 within desired limits.

The source of entrainment gas 134 is arranged to provide a flow of entrainment gas along the entrainment passage 132. The source of entrainment gas 134 may be a pump or a pressurised reservoir such as a gas cylinder. The entrainment gas is preferably air.

The entrainment passage 132 is in communication with the sampling cavity 120.

The entrainment passage 132 is arranged such that flow of entrainment gas along the entrainment passage 132 sucks exhaust gas from within the sampling cavity 120 into the entrainment passage 132.

For example, the flow of entrainment gas, being under pressure, may generate a first pressure in the entrainment passage 132 where it communicates with the sampling cavity 120. The sampling cavity 120 is at a second pressure, higher than the first pressure. Accordingly, a flow of gas from the sampling cavity 120 to the entrainment passage 130 can be produced.

Moreover, the flow of entrainment gas along the entrainment passage 132 can draw exhaust gas along the sampling tube 110 from the exhaust passage 10 via the sampling cavity 120 into the entrainment passage 132.

The entrainment passage 132 may create a pressure that is lower than the pressure in the sampling cavity 120 by including therein a venturi tube 130. As is known in the art, a venturi tube 130 has a constriction within which a pressure drop is created by the flow of gas through the constriction.

The entrainment passage 132 includes an outlet 135 in the exhaust passage 10. In this way, the mixture of entrainment gas and sampled exhaust gas can be delivered into the flow of exhaust gas.

In the embodiment of FIG. 1, the sampling cavity 120 is connected to the constriction for drawing the exhaust gas from the sampling cavity 120 into the flow of entrainment gas through the venturi tube 130 in the entrainment passage 132.

In the preferred embodiment shown in FIG. 1, the entrainment passage 132 includes a heat exchanger 136. For example, the heat exchanger 136 may be a coil of pipe.

The heat exchanger 136 extends into the exhaust passage 10. In this way, the heat exchanger 136 is arranged to receive heat from the exhaust gas. The heat exchanger can thereby provide a heated flow of entrainment gas along the entrainment passage 132. In particular, the flow of entrainment gas may be heated to the same temperature as the exhaust gas.

The source of span gas 140 includes a supply tube 142. The supply tube 142 delivers span gas into the monitoring apparatus upstream of the sampling cavity 120. In this way, span gas may be introduced into the sampling cavity 120 for the calibration of the sensors.

Preferably, the supply tube 142 extends through the exhaust passage 10 in order to introduce span gas into the sampling tube 110. For example, the supply tube 142 extends through the exhaust passage 10 in order to introduce span gas into the distal end of the sampling tube 110. By extending through the exhaust passage 10, the span gas within the supply tube 142 and within the sampling tube 110 can receive heat from the exhaust gas, so that the span gas is heated, for example, up to the temperature of the exhaust gas. In that way, the temperature of the monitoring apparatus 100 can be unaffected by the use of the span gas. Optionally, the supply tube 142 may include a heat exchanger (not shown) within the exhaust passage 10, such as a coiled section. However, a heat exchanger is not essential for receiving heat from the exhaust gas to thereby provide a heated flow of span gas, since the volume of span gas can be small.

The exhaust system 1 includes a controller 200 arranged to control the source of span gas 140 to introduce span gas into the sampling cavity 120. The controller 200 may also transform the measurement signals of the sensors into readable emission concentrations referenced to reference state, volume flows, and exhaust gas properties such as temperature and pressure. The controller 200 may also control the volume flow through the sampling cavity 120 through pressure, temperature readings and valve controls.

Whereas, it is conventional to calibrate this type of apparatus once, on installation, preferably, the controller 200 is arranged to introduce span gas at certain times during use of the exhaust system 1. That is, the monitoring apparatus 100 may be calibrated during use of the exhaust system 1. Specifically, span gas may be introduced into the sampling cavity 120 for the calibration of the sensors. In this way, “drift” of the sensor output over time may be lessened or avoided.

For example, calibration of the monitoring apparatus 100 may be carried out: intermittently; at predetermined times; at a particular frequency; and/or in a scheduled way in response to certain events (e.g., on start-up of the stationary source 2 or based upon temperature readings).

The sample gas pressure sensor 150 can provide an indication of the pressure required of span gas to be provided by the source of span gas 140 in order to provide a suitable flow into the sample cavity 120. That is, the source of span gas 140 is preferably arranged to provide span gas at a pressure greater than the pressure of the exhaust gas as sensed by the sample gas pressure sensor 180.

In embodiments in which one or more dust filters are provided within the holes of the sampling tube 110, it may be desirable to provide a flow of gas out of the sampling tube 110 into the exhaust passage 10 in order to clear the filters. Accordingly, the source of entrainment gas 134 may be connected to a filter cleaning tube 138 by suitable valving. The filter cleaning tube 138 may be arranged to provide a flow of the entrainment gas into the sampling tube 110 at a pressure exceeding the pressure in the exhaust passage 10 so as to flow out of the sampling tube 110 into the exhaust passage 10. More preferably, the filter cleaning tube 138 may include a heat exchanger 139 within the exhaust passage 10, such as a coiled section.

The exhaust system 1 described above, or similar exhaust systems, can be used in a method of monitoring exhaust gas from a stationary source 2.

A preferred embodiment of a method of monitoring exhaust gas from stationary comprises collecting a sample of exhaust gas from an exhaust passage 10 of the stationary source 2; delivering the sample of exhaust gas to a sampling cavity 120; and measuring the concentration of one or more airborne species within the sampling cavity using one or more sensors 122, 124.

In such a method, the sample of exhaust gas can be delivered to the sampling cavity 120 by drawing exhaust gas along the sampling tube 110 using a lower pressure region created by a flow of entrainment gas along an entrainment passage 132 in communication with the sampling cavity 120.

When reference is made to a sample of exhaust gas, this includes either a discrete volume or a continuous flow of gas collected from the exhaust passage 10.

After the sample of exhaust gas has been entrained into the entrainment gas, the method further comprises delivering the combined flow of sampled exhaust gas and entrainment gas into the exhaust passage 10.

Preferably, the method includes heating the entrainment gas before it is used to entrain the sample of exhaust gas. This can provide a heated flow of entrainment gas to be used to entrain the sampled exhaust gas. For example, the method may include passing the entrainment gas through the exhaust passage 10 (for example, via a heat exchanger 136) so that the entrainment gas receives heat from the exhaust gas. More preferably, the entrainment gas may receive heat from the exhaust gas so that the temperature of the entrainment gas is brought to, or closer to, the temperature of the exhaust gas.

The method can include the steps of monitoring the amount or concentration of one or more airborne species, such as one or more of NOx, NH₃, hydrocarbons (including CH₄, soot and its precursors), CO, O₂, and SOₓ.

The method may further include a calibration step. The calibration step comprises introducing span gas into the sampling cavity 120 and taking a measurement of the span gas with the sensors 122, 124. The span gas has known properties and so the measurement can be used to determine any error in the readings from the sensors 122, 124. For example, the determined error can be used to adjust subsequent measurements taken by the sensors 122, 124.

In a preferred method, calibration is carried out at certain times during use of the exhaust system 1. Specifically, span gas may be introduced into the sampling cavity 120 for the calibration of the sensors while exhaust gas is being exhausted through the exhaustion passage 10. The method may include intermittently introducing span gas into the sampling cavity during use of the stationary source 2 and synchronously carrying out calibration.

For example, in optional embodiments, span gas is introduced and calibration carried out at predetermined times, for example at a particular frequency and/or in accordance with a schedule. Alternatively, or in addition, span gas is introduced and calibration carried out in response to certain events. Those events may include: start-up of the stationary source 2; when certain temperature readings are taken; when certain error messages from the sensors 122 or 124 are attained; or when unusual readings (e.g. readings outside of predetermined expected limits) are observed.

The sample gas pressure sensor 180 can provide an indication of the pressure required of span gas to be provided by the source of span gas 140 in order to provide a suitable flow into the sample cavity 120. That is, the source of span gas 140 is preferably arranged to provide span gas at a pressure greater than the pressure of the exhaust gas as sensed by the sample gas pressure sensor 180.

In preferred embodiments, the span gas is introduced into the sampling tube 110. More preferably, the method includes introducing heated span gas into the sampling tube 110.

Preferably, the method includes the step of introducing span gas into the distal end of the sampling tube 110. In embodiments like this, the method can include heating the span gas with the exhaust gas. Thus, the span gas can be heated, for example, up to the temperature of the exhaust gas. This can provide a more accurate calibration procedure.

For example, the supply tube 142 may extend through the exhaust passage 10 in order to introduce span gas into the distal end of the sampling tube 110. The span gas can receive heat from the exhaust gas as it passes through each of the supply tube 142 and the sampling tube 110, so that the span gas is heated, for example, up to the temperature of the exhaust gas.

In embodiments in which one or more dust filters are provided within the holes of the sampling tube 110, the controller 200, or another controller, may be configured so as to control valving in order to direct a flow of entrainment gas out of the sampling tube 110 into the exhaust passage 10 in order to clear the filters. This may be carried out periodically and/or in response to an event such as a determination that the pressure measured by the sample gas pressure sensor 180 differs from the pressure measured by the exhaust gas pressure sensor 150 by more than a threshold amount.

The monitoring apparatus 100 described above may be provided as a single monitoring unit in order to be retrofit to an exhaust system 1 for a stationary source 2. The monitoring unit may comprise a body 15. Body 15 may, for example, be: a wall of a housing enclosing some or all of the components of the monitoring apparatus 100; a frame supporting some or all of the components of the monitoring apparatus 100, or a flange (a flange is shown in FIG. 1) to which the components of the monitoring apparatus 100 are mounted.

A method of retrofitting the monitoring unit may comprise making an aperture 12 in an exhaust passage 10 of a stationary source 2, and attaching the body 15 of the monitoring unit to the aperture 12.

For example, as shown in FIG. 1, the sampling tube 110 extends from a first side of the body 15.

The sampling cavity 120, the one or more sensors 122, 124, the source of entrainment gas 134, and the optional source of span gas 140 may be located on a second side of the body 15. The second side is opposite the first side.

The optional exhaust gas pressure sensor 150, the optional heat exchanger 136 of the entrainment passage 132, the optional supply tube 142, the optional heat exchanger 139 of the filter cleaning tube 138, and/or the outlet 135 of the entrainment passage 132 may extend from the same side of the body 15 as the sampling tube 110.

1. An exhaust system for a stationary source, the system comprising an exhaust passage and monitoring apparatus for monitoring exhaust gas, the monitoring apparatus comprising:
a sampling tube extending into the exhaust passage for collecting a sample of exhaust gas;
a sampling cavity in communication with the sampling tube; and
one or more sensors for measuring the concentration of one or more airborne species within the sampling cavity,
wherein the monitoring apparatus comprises suction generating apparatus in communication with the sampling cavity for drawing exhaust gas along the sampling tube into the sampling cavity.

2. The system according to claim 1, wherein:
the suction generating apparatus comprises a source of entrainment gas arranged to provide a flow of entrainment gas along an entrainment passage,
the entrainment passage is in communication with the sampling cavity for drawing exhaust gas along the sampling tube via the sampling cavity into the entrainment passage; and
the entrainment passage includes a heat exchanger extending into the exhaust passage for receiving heat from the exhaust gas to thereby provide a heated flow of entrainment gas along the entrainment passage.

3. The system according to claim 2, wherein the entrainment passage includes therein a venturi tube having a constriction to which the sampling cavity is connected for drawing the exhaust gas into the flow of entrainment gas.

4. The system according to claim 2, wherein the source of entrainment gas in communication with the entrainment passage is a pump or pressurised reservoir.

5. The system according to claim 2, wherein the entrainment passage includes an outlet in the exhaust passage for delivering the flow of entrainment gas into the flow of exhaust gas.

6. The system according to claim 1, wherein the sensors include one or more of: a NOx sensor; a NH₃ sensor; a sensor for acidic species; or a sensor for unburnt species.

7. The system according to claim 1, further comprising a source of span gas upstream of the sampling cavity, wherein the system includes a controller arranged to control the source of span gas to introduce span gas into the sampling cavity during use of the exhaust system.

8. A system according to claim 7, wherein the source of span gas is arranged to introduce span gas into the sampling tube.

9. The system according to claim 8, wherein the source of span gas is delivered into the sampling tube via a span gas heat exchanger extending within the exhaust passage for receiving heat from the exhaust gas to thereby provide a heated flow of span gas.

10. The system according to claim 7, further comprising an exhaust gas pressure sensor for sensing the pressure of the exhaust gas, wherein the source of span gas is arranged to provide span gas at a pressure greater than the pressure of the exhaust gas.

11. A method of monitoring exhaust gas from a stationary source, comprising:
collecting a sample of exhaust gas from an exhaust passage of the stationary source;
delivering the sample of exhaust gas to a sampling cavity; and
measuring the concentration of one or more airborne species within the sampling cavity using one or more sensors,
wherein the method is characterised by drawing exhaust gas along the sampling tube into the sampling cavity.

12-20. (canceled)

21. A monitoring apparatus for attachment to an exhaust passage of a stationary source, comprising:
a body having a first side and a second side, the body arranged to be located over an aperture with the first side facing the aperture and the second side facing away from the aperture;
a sampling tube for collecting a sample of exhaust gas, extending from the first side of the body;
a sampling cavity on the second side of the body, the sampling cavity in communication with the sampling tube;
one or more sensors for measuring the concentration of one or more airborne species within the sampling cavity; and
suction generating apparatus on the second side of the body and arranged to draw exhaust gas along the sampling tube into the sampling cavity.

22. The apparatus according to claim 21, wherein:
the suction generating apparatus comprises a source of entrainment gas arranged to provide a flow of entrainment gas along an entrainment passage;
the entrainment passage is in communication with the sampling cavity for drawing exhaust gas along the sampling tube via the sampling cavity into the entrainment passage; and
the entrainment passage includes a heat exchanger extending from the first side of the body for receiving heat from exhaust gas to thereby provide a heated flow of entrainment gas along the entrainment passage.

23. The apparatus according to claim 22, wherein the entrainment passage includes therein a venturi tube having a constriction to which the sampling cavity is connected for drawing the exhaust gas into the flow of entrainment gas.

24. The apparatus according to claim 22, wherein the source of entrainment gas in communication with the entrainment passage is a pump or pressurised reservoir.

25. The apparatus according to claim 22, wherein the entrainment passage includes an outlet for delivering the flow of entrainment gas into the flow of exhaust gas, the outlet extending from the first side of the body.

26. The apparatus according to claim 21, wherein the sensors include one or more of: an NOx sensor; an NH₃ sensor; a sensor for acidic species; a sensor for unburnt species.

27. The apparatus according to claim 21, further comprising a source of span gas upstream of the sampling cavity, wherein the apparatus includes a controller arranged to control the source of span gas to introduce span gas into the sampling cavity.

28. The apparatus according to claim 21, wherein the source of span gas is arranged to introduce span gas into the sampling tube.

29. The apparatus according to claim 28, wherein the source of span gas is delivered into the sampling tube via a span gas heat exchanger for receiving heat from the exhaust gas to thereby provide a heated flow of span gas, wherein the span gas heat exchanger extends from the first side of the body.

30. The apparatus according to claim 27, further comprising an exhaust gas pressure sensor for sensing pressure on the first side of the body.

31. A method of fitting the apparatus of claim 21 to an exhaust passage of a stationary source, comprising: making an aperture in the exhaust passage, and attaching the first side of the body over the aperture such that the sampling tube extends into the exhaust passage.
